(19) United States
(12) Patent Application Publication
(10) Pub. No.: US 2007/0160655 A1
(43) Pub. Date: Jul. 12, 2007

(54) Hydroxyamate-containing materials for the inhibition of matrix metalloproteinases

(76) Inventors: Michael V. Sefton, Toronto (CA); Michael H. May, Brantford (CA); Gary A. Skarja, Toronto (CA); Allison Brown, Toronto (CA)

Correspondence Address: RICHES, McKENZIE & HERBERT LLP, SUITE 1800, 2 BLOOR STREET EAST, TORONTO, M4W 3J5, CA

(21) Appl. No.: 11/714,730

(22) Filed: Mar. 7, 2007

Related U.S. Application Data
(63) Continuation-in-part of application No. 10/420,725, filed on Apr. 23, 2003.

(51) Int. Cl. A61K 31/785; C08F 8/30

(52) U.S. Cl. 424/445; 424/78.27; 525/327.6

(57) ABSTRACT

Therapeutic polymers containing a hydroxamate group that preferentially binds to active forms of a matrix metalloproteinases, thus inhibiting their enzymatic activity. The implantation of such material modifies tissue turnover and remodelling in its vicinity.

### RELATED APPLICATIONS

This is a continuation-in-part application of U.S. patent application Ser. No. 10/420,725 filed on Apr. 23, 2003 for HYDROXYAMATE-CONTAINING MATERIALS FOR THE INHIBITION OF MATRIX METALLOPROTEINASES

### FIELD OF THE INVENTION

This invention relates to therapeutic polymers containing a hydroxamate (HX) group that bind, and thus inhibit, zinc-containing enzymes, such as matrix metalloproteinases (MMPs). By inhibiting MMPs, the material, once implanted, inhibits tissue remodeling in its vicinity.

### BRIEF DESCRIPTION OF THE DRAWINGS

FIG. 1 illustrates the cysteine switch mechanism for the activation of MMPs.

FIG. 2 illustrates the chemical structure of the hydroxamate functional group.

FIG. 3 illustrates the inhibition of the degradation of gelatin tubes implanted in mice in the presence of hydroxamate-derivatized beads.

FIG. 4 illustrates the effects of the initial MAA content of cross-linked PMMA-MAA beads on the degree of hydroxamate derivatization.

FIG. 5 illustrates hydroxamate-derivatized beads with differing base surface MAA content stained with ferric chloride.

FIG. 6 illustrates fluorescence of FITC-labeled gelatin degradation products after incubation with MMP-2 and hydroxamate derivatized (right panel) versus underivatized (left panel) PMMA-MAA beads (63% initial MAA content).

FIG. 7 illustrates the NMR spectrum for hydroxamate monomer after purification to 95%.

FIG. 8 illustrates the MMP-8 concentration and MMP activity HX bead dose response curves for pro-(inactive) MMP-8 and catalytic domain MMP-8.

FIG. 9 illustrates the MMP-8 concentration and MMP activity HX bead dose response for human chronic wound fluid.

FIG. 10 illustrates dose-dependent inhibition of MMP activity by HX beads towards several subclasses of MMPs.

FIG. 11 illustrates the reduction of MMP activity in human arthritic synovial fluid samples following incubation with HX beads.

FIG. 12 illustrates the activation of MMP-3 by heat treatment. The slopes of the absorbance vs. time curves are proportional to MMP activity.

FIG. 13 illustrates the effect of MMP-3 activation on HX bead efficacy. Reductions in MMP-3 concentration due to HX bead binding were determined by ELISA.

FIG. 14 illustrates the activation of recombinant MMP-8 by treatment with MMP-3.

FIG. 15 illustrates the effects of MMP-8 activation on HX bead efficacy. Reductions in MMP-8 concentration due to HX bead binding were determined by ELISA.

### BACKGROUND OF THE INVENTION

The following definitions and acronyms will be used in this specification:

| | |
|---|---|
| HX | hydroxamate |
| MMPs | matrix metalloproteinases or matrixins |
| TIMPs | tissue inhibitors of metalloproteinases |

Matrix metalloproteinases (MMPs), also called matrixins, are neutral zinc-dependent endopeptidases with substrate specificity for most extracellular matrix molecules, including collagens, gelatins, fibronectin, laminin and proteoglycan. To date, over 25 MMPs have been identified with many of them possessing a common name indicating the vulnerable extracellular matrix component: collagenases 1-4, gelatinases A-B, stromelysins 1-3, matrilysin, and enamelysin.

Cells do not constitutively express most MMPs in vivo; rather, growth factors, hormones, inflammatory cytokines, cell-matrix interactions and cellular transformation regulate their expression transcriptionally. Although the secretory granules of neutrophils and eosinophils are known to store MMP-8 and MMP-9, most cell types normally synthesize very low quantities of MMPs.

The MMPs share some common structural characteristics that include a signal sequence, an amino-terminal pro-peptide domain, a catalytic zinc binding domain, a proline-rich hinge region, and a carboxy-terminal hemopexin-like domain. The pro-peptide domain of all MMPs contain a highly conserved amino acid sequence with a single cysteine residue which is critical for maintaining enzyme latency. This residue forms a bond with the zinc atom located within the active site of the catalytic domain forming the basis for the “cysteine switch model of activation”: When the cysteine is “on” the zinc, the active site is blocked and enzyme activity is “off”. Conversely, when the sulfhydryl group of the cysteine is dissociated from the zinc atom, the active site is exposed, leading to activation [Van Wart et al., 1990].

Since all MMPs are secreted as inactive zymogens, physiological activation is an important control point in matrix degradation. Physiologic activators of MMPs include proteolytic enzymes such as plasmin, urokinase type plasminogen activator (uPA), tissue-type plasminogen activator (tPA) and also reactive oxygen species released from activated inflammatory cells [Chakraborti et al., 2003]. In addition, interaction between different MMPs can lead to activation. For example, MMP-8 can be activated by MMP-3 [Knauper et al., 1993], MMP-7 [Balbin et al., 1998], MMP-10 [Knauper at al., 1996], and MMP-14 [Holopainen et al., 2003].

A variety of reagents have been utilized for the activation of MMPs. These include non-proteolyic, chemical reagents, as well as physiologic, proteolytic enzymes. Depending upon the nature of the activator, several pathways towards activation are possible. In general, the resulting state of enzyme activation is dependent upon the extent of active zinc site exposure.

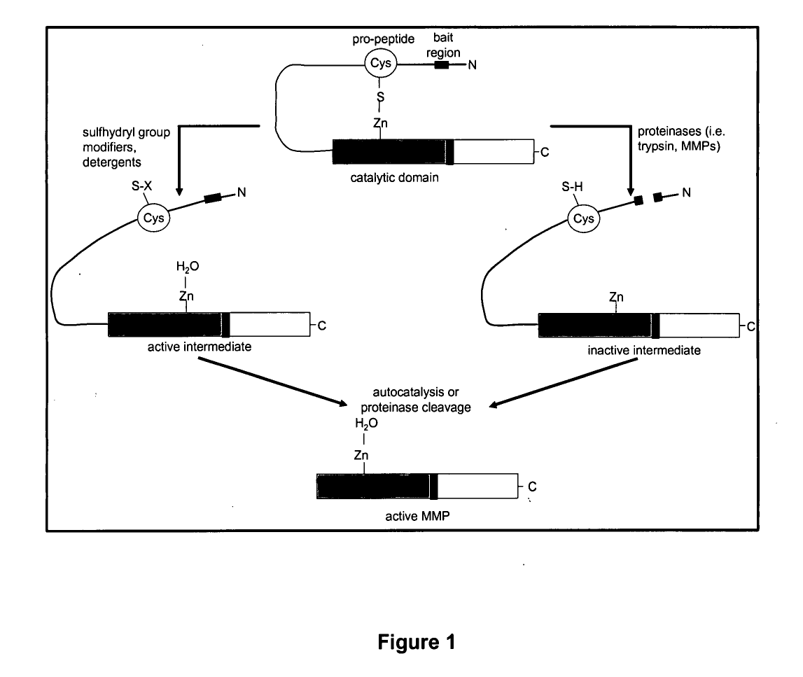

With non-proteolytic reagents (left pathway of FIG. 1), the first step towards activation involves the disruption of the interaction between the cysteine residue and the zinc ion. This can be achieved by detergent treatment, which induces a conformational change in the pro-peptide domain, or by chemical modification to the cysteine residue with sulfhydryl group modifiers (ie. organomercurials such as aminophenyl mercuric acetate (APMA), sulfhydryl alkylating agents, oxidants and heavy metal ions). Following this destabilization of the cysteine-zinc interaction, the zinc site is partially exposed, leading to the generation of an intermediate enzyme form, which possesses some activity (typically 15-25% of maximum potential activity). This step is then followed by autocatalytic cleavage, in which the pro-peptide domain is completely removed and the enzyme processed to the final active form [Suzuki et al., 1990]. MMPs activated via this pathway generally possess a terminal enzymatic activity which is 30-40% of maximum potential activity (Knauper et al 1991).

MMP activation with physiologic, proteolytic enzymes such as trypsin, plasmin, and MMPs proceeds via an alternative route. These enzymes cleave a portion of the MMP pro-peptide in a bait region ahead of the cysteine residue to yield an intermediate, yet still inactive product (right pathway of FIG. 1). This intermediate can then undergo one of two routes towards activation. It may undergo autocatalytic processing with the loss of the entire pro-peptide domain containing the cysteine switch residue, as occurs with MMP-1 [Stricklin et al., 1983] or a second N-terminal cleavage by trypsin may occur within the intermediate form just ahead of the cysteine residue, resulting in conversion to the active form, as occurs with MMP-8 [Knauper et al., 1990].

A third, effective physiologic activator of the collagenases MMP-1 and -8 is active MMP-3. Incubation of the collagenases with activated MMP-3 generates a single cleavage site in the pro-peptide domain leading to direct conversion to a “superactivated” final form, which is approximately 3.5 fold more active than the other two species described for trypsin and APMA treatment [Knauper et al., 1993].

Extracellular matrix degradation is a normal event in the physiological remodeling associated with morphogenesis, reproduction, and in such growth and maintenance processes as cell migration, angiogenesis, and tissue regeneration. During inflammation and in several disease situations, however, excess MMPs degrade the surrounding proteinaceous matrix, which results in the destruction or weakening of connective tissue, unregulated cell migration/invasion, and tissue fibrosis. For example, connective tissue weakening or destruction results in diseases such as rheumatoid arthritis, osteoarthritis, chronic periodontis, and arterial and cardiac aneurysm. Accordingly, MMP inhibitors have been used to treat osteoporosis, osteoarthritis, human chronic periodontal disease [Ashley, 1999] and various types of aneurysms [Thompson and Baxter, 1999; Prescott et al., 1999].

Chronic wounds take months or years to heal due, in part, to high levels of MMPs that degrade the newly formed matrix even as it is synthesized. The role of MMPs in the poor healing of gastric and skin ulcers [Trengove et al, 1999; Saarialho-Kere, 1998] has been studied extensively. This work has not translated into significant research into the use of MMP inhibitors to treat chronic wounds [Parks et al., 1998], despite evidence that administration of GM6001, a collagenase inhibitor, increased the strength of linear incision rat skin wounds [Witte et al., 1999].

Angiogenesis or vasculogenesis of tumours and the formation of metastases require cell migration and invasion, which are enabled by the release of pro-MMPs. Various MMP inhibitors are being evaluated clinically for their anti-tumoral and antimetastatic potential [Drummond et al. 1999; Shalinsky et al., 1999]. Furthermore tissue remodeling occurs secondary to secretion or expression of MMP's. Thus blood vessels associated with wound repair are resorbed or ischemic tissue is destroyed by MMP action.

The activity of MMPs is essential for many of the processes involved in atherosclerotic plaque formation (infiltration of inflammatory cells, angiogenesis, and smooth muscle cell migration and proliferation). Elevated levels of MMPs are expressed in human atherosclerotic plaque and at the sites of aneurysm [Prescott et al., 1999]. Furthermore, matrix degradation by MMPs may cause the plaque instability and rupture that leads to the clinical symptoms of atherosclerosis. Recent studies using synthetic MMP inhibitors have highlighted the potential approach of MMP inhibition to treat atherosclerosis [George, 2000].

Studies focussed on elucidating the role of MMPs in the progression of chronic wounds and diseases such as atherosclerosis have primarily reported upon the upregulation of overall MMP levels during pathogenesis. More recently, investigations have focussed on distinguishing between the relative contributions of pro-MMP production and actual MMP activation. Numerous examples in the literature have demonstrated that an increase in the proportion of active relative to inactive enzyme forms is a primary contributing factor to the progression of chronic inflammation and excessive matrix destruction.

For example, several studies have demonstrated elevated activities of MMPs in the wound fluid and tissue of chronic, non-healing compared to acute, healing wounds, suggesting that the sustained inflammatory response associated with the chronic wound environment leads to an alteration in not only the total amount of MMPs expressed, but also the proportion existing in the active form [Lobmann et al., 2005; Lobmann et al., 2002; Nwomeh et al., 1999; Trengrove et al., 1999; Wysocki et el., 1993; Yager et al., 1996].

In the context of arthritis, a similar role for activated MMPs in cartilage degradation has been demonstrated. Elevated concentrations and activities of several MMPs including MMP-1, MMP-3, MMP-8 and MMP-13, as well as aggrecanase (another metalloproteinase) have been identified in the synovial fluid of osteoarthritis and rheumatoid arthritis patients. [Amer, 2002; Marini et al., 2003; Sandy & Verscharen, 2001; Tchetverikov et al., 2005; Tchetverikov et al., 2004, Yoshihara et al., 2000].

There is also accumulating evidence that an increase in the proportion of active MMPs is associated with the progression of restenosis following vascular interventions such as balloon angioplasty or intra-coronary stenting, for the treatment of coronary artery disease. In contrast to the non-diseased vessel wall, which constitutively expresses only pro-(inactive) MMP-2, injured or atherosclerotic arteries demonstrate a dramatic increase in MMP-2 activity. This occurs in conjunction with induced expression of MMPs-3, -7, -9, -12, and -13. An increase in the proportion of the active form of these MMPs is also observed [Galis et al., 2002; Lijnen et al., 2004].

Given these reports, an attractive strategy towards restoring the balance of matrix synthesis and destruction in these chronic, pathological conditions may be through the selective, targeted binding of active MMPs leading to inactivation and a progress towards healing.

MMP activity is inhibited non-specifically by α₂-macroglobulin, a serum protein, and specifically in tissue by TIMPs, tissue inhibitors of metalloproteinases. The most popular approach to reducing MMP levels in tissue pharmacologically is the use of chelating agents such as antibiotics, tetracycline, thiols, carboxyalkyl, phosphonamidates and hydroxamates. These agents inactivate MMPs by binding the zinc at the active center of the enzymes. The hydroxamates are the most popular synthetic means of inhibiting MMP activity. With multiple point attachments, they behave like a molecular magnet for zinc.

Numerous soluble hydroxamates (e.g., Batimastat™, Marimastat™, Galardin™, Ro31-9790™) have been designed to broadly inhibit all MMPs, or inhibit one or more varieties of the same basic enzyme (e.g., the three collagenases) without any effect on related enzymes (e.g., stromelysin or gelatinase). The primary reason for making these inhibitors soluble is to enable systemic delivery. Modifications to the basic hydroxamate functionality have focused on reducing toxicity, increasing solubility, improving bioavailability, increasing stability and imparting specificity. Toxicity and specificity are concerns because MMPs play important roles in normal biological function and systemic delivery of broad-spectrum inhibition can interfere with their normal function. No consensus has yet been reached on whether MMP inhibitors should act on many MMPs or be highly specific. Typically, specificity is achieved by adding specific peptide sequences to molecules containing the hydroxamate group.

Currently, soluble hydroxamate compounds have been prepared with IC₅₀ between 1 and 5 μM for MMP-1, -3 and -7 [Chen et al., 1996]. Some hydroxamates such as Marimastat™ [Wojtowicz-Praga et al., 1996] and Trochate™ [Lewis et al., 1997] are now in clinical trials.

The MMPs are a subclass of a larger (that is, greater than 200) set of proteases that depend on zinc for their catalytic activity. Some of these proteases have similar binding pockets as the MMPs, so it is possible that the inhibitors of MMPs may also inhibit the activity of other zinc proteases [Woessner, 1998].

Hydroxamate-containing polymers that are capable of reversibly binding a number of metal ions (e.g. V⁵⁺, Fe³⁺, Zn²⁺, Au³⁺, UO²⁺) have been proposed for use in several industrial and laboratory applications. These include the removal of metals from water [Vernon and Eccles, 1976], recovery of precious metals and metal catalysts in industrial processes [Vernon and Zin, 1981] and chromatographic separation [Kamble and Patkar, 1994]. As far as we can determine, no hydroxamate-containing polymers have been proposed to inhibit the activity of the Zn-containing MMPs. In fact, all known references to hydroxamate-containing polymers for biomedical applications deal with the chelation of iron or inhibition of nickel-containing urease. Applications include the treatment of iron overload from poisoning or transfusion-dependent anemias [Domb et al., 1992; Winston et al. 1985; Winston et al., 1986, Horowitz et al., 1985; Gehlbach et al, 1993], the coating of medical devices against coagulation [Domb et al., 1992], the in vivo inhibition of urease to reduce the incidence of infection-induced urinary stones [Domb et al., 1992], the widespread protection of tissues from iron-catalyzed oxygen free radical damage [Panter et al., 1992], protection from oxygen damage applied to the treatment of chronic wounds [Wenk et al., 2001], and the use of a hydroxamate-derivatized PEG as a renal magnetic resonance contrast agent [Duewell et al., 1991].

Two approaches have been employed to produce hydroxamate-containing polymers: 1) (co)polymerization of vinyl monomers bearing hydroxamate groups and 2) post-polymerization modification of polymer functional groups (e.g. carboxylic acid, ester, nitrile, amide) to generate hydroxamate groups.

Hydroxamate-bearing monomers were synthesized [Iskander et al. 2000] by reacting methacryloyl chloride (acid chloride of methacrylic acid) with hydroxylamine or various hydroxyalkyl hydroxamates under basic conditions. These monomers were then used to generate homo- and co-polymers by free radical polymerization processes. A number of researchers have generated hydroxamate-containing polymers via post-polymerization derivatization. Typically, the functionality is introduced via a nucleophilic displacement of polymer functional groups by hydroxylamine or hydroxylamine derivatives. Polymers derivatized in this way include polyacrylates [Kern and Schulz, 1957], polyacrylamide [Domb et al, 1992], polyacrylonitrile [Schouteden, 1958], and polyoxetanes [Xu et al, 1999]. Hydroxamate functionality was also imparted to polyethylene glycol [Duewell et al, 1991], various polysaccharides [Hallaway et al., 1989], and cellulose [Feldhoff, 1992] by activating hydroxyl groups for subsequent reaction with desferrioxamine-B, a tri-hydroxamic acid. Alternatively, polyacrylics may be directly reacted with hydroxylamine at high temperatures [Sparapany, 1989] or dehydrated to the corresponding anhydrides followed by reaction with hydroxylamine to generate hydroxamate functionality [Huffman, 1989].

The purpose of the current application is to describe a novel polymeric hydroxamate MMP inhibitor which preferentially binds to the active form of MMPs. The invention is not specific for particular MMP types, but rather targets any enzyme possessing the conserved catalytic zinc motif. Due to the solid nature of this inhibitor, binding affinity to MMPs will depend upon accessibility of the inhibitor surface to the catalytic zinc site. The degree of zinc site exposure is directly related to the extent of pro-peptide processing by the activating agent. As a consequence of this mechanism, the affinity for binding to inactive MMPs would be greatly reduced due to the steric hindrance imparted by an intact pro-peptide domain.

The ability of this polymer to provide preferential binding to active forms of MMPs in the local tissue environment is advantageous because it specifically targets one stage in the MMP regulatory cascade, namely that directly preceding matrix degradation. In addition, selective binding reduces the risk of over inhibition which would delay healing by preventing a healthy rate of tissue turnover and essential processes such as cell migration and angiogenesis.

### SUMMARY OF THE INVENTION

It is an object of the present invention to synthesize polymers containing HX groups which have the same biological effect as soluble hydroxamate MMP inhibitors, but that have many novel advantages. These materials, which combine the physiochemical properties of polymers with novel biological activity, are referred to as therapeutic polymers.

It is a further object of this invention to provide a novel polymer that inhibits the activity of biological species containing divalent metal ions, more specifically zinc-containing proteases and in particular, the matrix metalloproteinases, which are responsible for a variety of medical disorders when over-expressed.

It is a further object of this invention to provide a novel polymer which preferentially binds to the active form of MMPs, lowering their enzymatic activity in solution.

It is a still further object of this invention to provide a novel polymer with MMP binding affinity which is dependent upon the degree of enzyme activation. This is in turn dependent upon the extent of catalytic zinc site exposure, as a result of pro-peptide processing.

It is a still further object of this invention to provide a novel polymer which binds the inactive form of MMPs with reduced affinity compared to active forms. This is a consequence of steric hindrance imparted by an intact pro-peptide domain and limited availability of the catalytic zinc towards the binding interaction with the polymer.

It is a further object of this invention to provide a novel polymer which binds to all subclasses of active MMPs. This is enabled by the fact that all MMPs contain a conserved zinc binding motif within the catalytic domain of the molecule.

It is a further object of this invention to provide a novel polymer which preferentially binds active MMPs in multi-protein physiologic solutions, thereby reducing overall solution MMP activity.

It is still a further object of this invention to provide an MMP inhibitor that can be formed into various constructs and geometries, or incorporated into various medical devices.

It is a further object of this invention to provide a novel MMP inhibitor whose activity is localized to a specific tissue or site in the body. As a polymeric material, the inhibitor may remain insoluble or be formed in a way that restricts its movement or clearance from the site of application.

It is a still further object of this invention to provide an MMP inhibitor that has improved bioavailability for a specific dose and a desired length of time. Doses can be lower and administered less frequently because the inhibitor acts locally and persists locally. The duration of inhibition can be varied by changing the properties of the polymer (e.g., degradation, porosity, composition, geometry and size).

It is another object of this invention to provide a novel polymeric MMP inhibitor that is less toxic than the small, soluble MMP inhibitors. Systemic toxicity is reduced because the inhibitor acts locally. Local toxicity is reduced because lower dosages can be used, since clearance from the tissue is not significant. In addition, the inhibitor is a large M.W., insoluble synthetic polymer that cells cannot internalize or metabolize easily.

It is another aspect of this invention to provide a novel polymeric MMP inhibitor that requires lower dosing regimes than non-specific, localized inhibitors which do not distinguish between active and inactive MMP forms. This object is enabled by the fact that the majority of MMPs within the body are present in their inactive form. By inhibiting the smaller, active constituent of localized MMPs within a tissue, this novel polymer provides a more efficient and cost-effective method for inhibiting matrix destruction.

It is a further object of this invention to provide a built-in control mechanism for reducing the risk of overdose. Selective binding to active MMP forms attenuates excessive matrix degradation while preserving inactive MMP forms which are integral to the regulation of normal physiologic processes

It is another object of this invention to provide an MMP inhibitor that is stable. This object is enabled by the fact that the inhibitor is an insoluble polymer, which is not degraded or metabolized easily by the body. In some situations a degradable HX polymer will be desirable, but in such cases, degradation can be controlled.

It is a further object of the invention to provide a novel method of removing MMPs in a safe and controlled manner. MMP-saturated constructs made from the non-degradable HX polymer can be removed by explantation or other means. A degradable version of the HX polymer would eventually become soluble and be cleared by the body after achieving its therapeutic purpose.

It is a further object of this invention to provide a method of derivatizing carboxylic-containing polymers to hydroxamic acid by a mixed anhydride intermediate (e.g., to make microbeads, nanoparticles and films).

It is a further object of this invention to provide a method of synthesizing a polymerizable hydroxamic acid unit by a mixed anhydride intermediate.

To this end, in one of its aspects, the invention provides a therapeutic polymer containing a hydroxamate group for preferential binding to an active form of a matrix metalloproteinase.

In another of its aspects, the invention provides a medical device for the inhibition of matrix metalloproteinases which comprise a therapeutic polymer containing a hydroxamate group for preferentially binding an active form of a matrix metalloproteinase.

In still another of its aspects, the invention provides surface modified derivatizable polymers containing a hydroxamate group for preferentially binding an active form of a matrix metalloproteinase.

In yet another of its aspects, the invention provides a surface modified derivatizable polymer containing a hydroxamate group wherein the matrix metalloproteinase has been activated by an activating agent which is either physiologic or non-physiologic in nature.

A further aspect of the invention provides a hydroxamate group containing polymer synthesized by copolymerizing a polymerizable monomer containing a hydroxamate group with a comonomer.

In still another of its aspects, the invention provides surface modified cross-linked polymethacrylic acid-co-methyl methacrylate beads containing a hydroxamate group.

A further aspect of the invention provides polymerizable therapeutic monomers containing a hydroxamate group.

A further aspect of the invention provides a therapeutic polymer containing a derivatizable polymer with a hydroxamate containing group grafted thereon.

In yet another of its aspects, the invention provides a therapeutic polymer for slowing, preventing or reversing tissue remodelling and destruction comprising a therapeutic polymer containing a hydroxamate group.

A further aspect of the invention provides a therapeutic polymer for controlling inflammation comprising a therapeutic polymer containing a hydroxamate group.

A yet further aspect of the invention provides a therapeutic polymer for restricting cell migration comprising a therapeutic polymer containing a hydroxamate group.

A still further aspect of the invention provides beads for slowing preventing or reversing tissue remodelling and destruction comprising a therapeutic polymer containing a hydroxamate group.

In yet another of its aspects, the invention provides beads for controlling inflammation comprising a therapeutic polymer containing a hydroxamate group.

A still further aspect of the invention provides beads for restricting cell migration comprising a therapeutic polymer containing a hydroxamate group.

In another of its aspects, the invention provides novel wound care products such as dressings, creams and ointments in which therapeutic polymers are incorporated.

A further aspect of this invention provides novel wound care products such as dressings, creams and ointments in which hydroxamate containing therapeutic polymers are incorporated.

In yet another of its aspects, the invention provides a wound care product which comprises a therapeutic polymer containing a hydroxamate group for preferential binding to an active form of a matrix metalloproteinase.

In yet another of its aspects, the invention provides a thermoreversible gel in which hydroxamate beads are incorporated, which gel may be applied to a wound as a liquid and then removed by washing with cool saline.

In yet a further aspect, the invention provides a thermoreversible gel in which hydroxamate beads are incorporated, which thermoreversible gel comprises a copolymer and a solvent, the copolymer having the structure A(B)n, wherein A is soluble in the solvent, B is convertible between soluble and insoluble in the solvent depending on an environmental condition, and n is greater than 1, the gel being convertible from liquid to gel under an environmental condition wherein B is insoluble.

A further object of the invention is to provide a wound dressing which comprises a thermoreversible gel in which hydroxamate beads are suspended.

A yet further object of the invention is to provide a wound dressing which comprises a thermoreversible gel which comprises a copolymer and a solvent, the copolymer having the structure A(B)n, wherein A is soluble in the solvent, B is convertible between soluble and insoluble in the solvent depending on an environmental condition, and n is greater than 1, the gel being convertible from liquid to gel under an environmental condition wherein B is insoluble, in which hydroxamate beads are suspended.

### DETAILED DESCRIPTION OF THE INVENTION

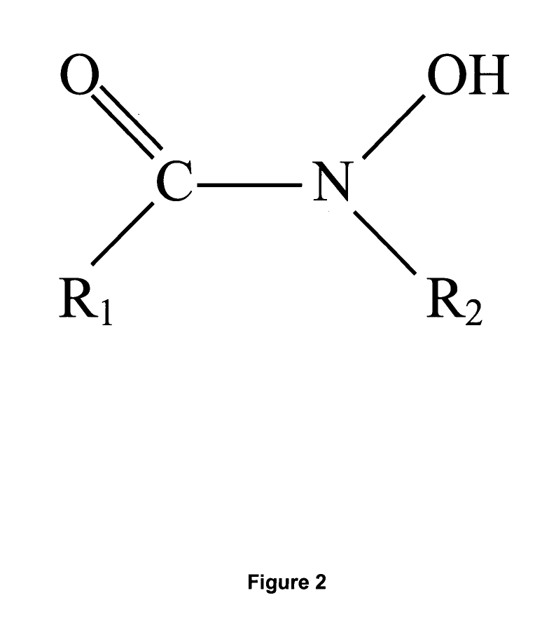

HX polymer is synthesized by surface modification of cross-linked polymethacrylic acid (PMAA)-co-methyl methacrylate (MAA) beads (resulting in a novel composition of PMAA-MMA-HX). In the example, with reference to FIG. 2, R1 represents the polymer main chain and R2 represents hydrogen. This method results in beads that are not soluble, but are useable as such; the surface modification method can be applied to other shapes, but the materials will need to be in their final form prior to modification.

Polymerizable HX monomer was synthesized. This monomer can be used to synthesize an HX homopolymer or copolymerized with any other suitable comonomers to produce polymers with a variety of properties. These polymers are suitable for coating other materials (e.g., stainless steel) or ones made into a solid material after conventional thermoplastic processing (moulding, extrusion, etc.) or beads or nanoparticles made by spray drying, solvent evaporation or any other conventional polymer processing method. In the example, with reference to FIG. 2, R1 represents CH₂═C—CH₃ and R2 represents hydrogen.

HX homopolymer synthesized from the HX monomer can also be grafted onto any derivatizable polymer to produce additional MMP-inhibiting polymers. Small beads of HX polymer may be injected in the vicinity of diseased or damaged tissue. Alternatively HX polymer can be incorporated into devices in contact with tissue.

The hydroxamate beads may be incorporated into a thermoreversible gel that can be applied to a wound as a liquid and then removed by washing with cool saline. An example of such thermoreversible gel is disclosed in PCT published application serial number PCT/CA01/00325 (publication number WO 01/68768) filed on Mar. 15, 2001 in the name of Cheng and Lin, the specification of which is incorporated herein by reference. Thermoreversible gels undergo structural changes in response to changes in the environment. Within the composition, the copolymer undergoes a phase transition from liquid to gel in response to changes in an environmental parameter such as for example temperature, pH, ionic strength of the composition or combinations of these parameters.

The thermoreversible gel can be used as a protective coating for a wound. In this embodiment, the hydroxamate beads are incorporated into the gel itself, which is then applied to the wound as a liquid. The gel is then removed by washing with a cool saline. One example of a thermoreversible gel comprises a copolymer and a solvent, the copolymer having the structure A(B)n, wherein A is soluble in the solvent, B is convertible between soluble and insoluble in the solvent depending on an environmental condition, and n is greater than 1, the composition being convertible from liquid to gel under an environmental condition where B is insoluble. The environmental condition to conversion from liquid to gel may be temperature, pH, ionic strength and a combination thereof.

In the preferred structure of the gel, A is polyethylene glycol (PEG), polyvinyl pyrrolidone, polyvinyl alcohol, polyhydroxyethylmethacrylate, and hyaluronic acid, and B is poly-N-isopropyl acrylamide (PNIPAAm), hdroxypropylmethyl cellulose and other methyl cellulose derivatives, poly(thylene glycol vinyl ether-co-butyl vinyl ether), polymers of N-alky acrylamide derivatives, poly(amino acid)s, peptide sequences, poly(methacryloy L-alanine methyl ester), poly(methacryloy L-alanine ethyl ester) and nitrocellulose. The copolymer may be present in the solvent at a level from 5 to 50% by weight, preferably, from 10 to 25% by weight. Also, the integer n may represent 2, 4 or 8 with the preferred embodiment being greater or equal to 4.

In a specific preferred embodiment of the gel, the letter A represents polyethyleneglycol (PEG) and B represents poly-N-isopropyl acrylamide (PNIPPAAm) and the solvent is aqueous.

This gel may be formed by a process comprising the steps of: (i) forming a copolymer having the structure A(B)ₙ, wherein A is soluble in a solvent of interest, B is convertible between soluble and insoluble in the solvent depending on an environmental condition, and n is greater than 1; (ii) solubilizing said copolymer in the solvent in an amount adequate to convert the composition from liquid to gel under an environmental condition where B is insoluble.

### EXAMPLES

### Example 1

### Surface Modification

Crosslinked poly(methyl methacrylate-co-methacrylic acid) (PMMA-MAA) beads were suspended in a suitable organic solvent (e.g. DMF, THF, diethyl ether) at approximately 10% wt/vol and allowed to equilibrate in solvent for at least 30 min at 0° C. while stirring. A 100% molar excess of N-methyl morpholine and chloroformate, relative to the MAA content of the beads, was added to the bead suspension. The reaction proceeded at 0° C. for 30 min. The beads were filtered from suspension and washed with DMF. The beads were transferred to a vessel containing a 100% molar excess of hydroxylamine solution in water and the reaction proceeded at ambient temperature for at least 1 hour. The beads were then filtered and washed with water, 0.1 M HCl, again with water, and then dried at 55-60° C.

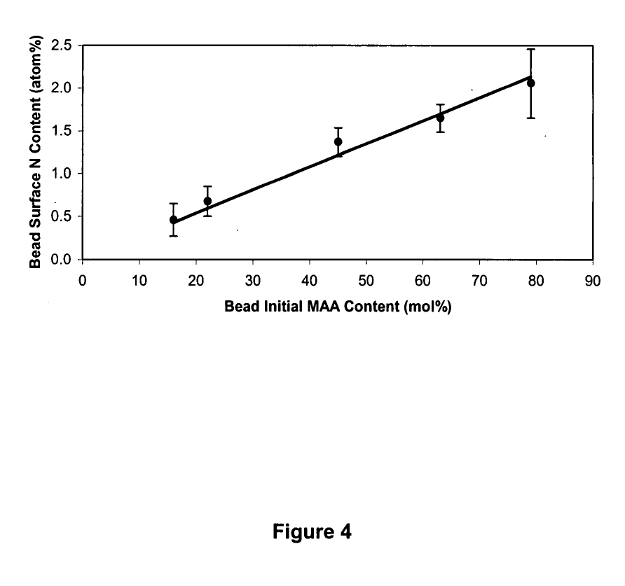

FIG. 4 shows that the hydroxamate content (as indicated by nitrogen content) of the copolymer beads may be varied in this process by altering the acid content of the base copolymer from 15 to 80 mol % MAA.

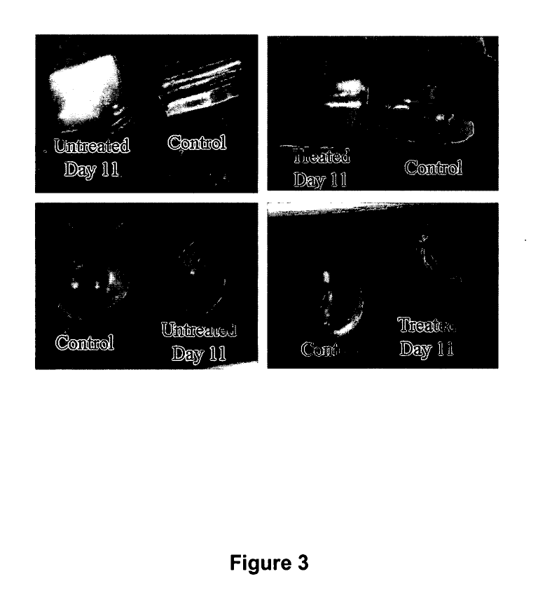

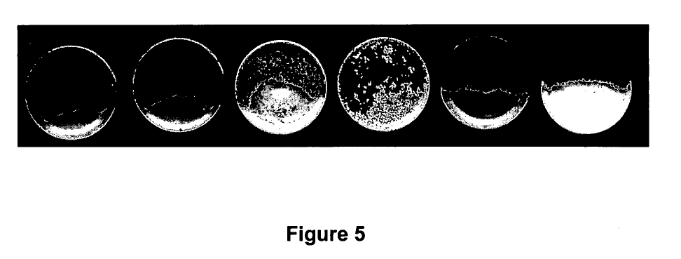

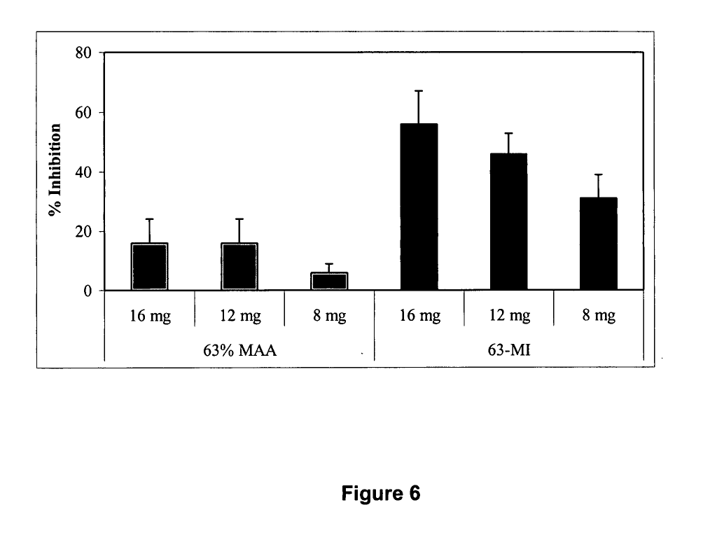

Ferric chloride stains hydroxamate groups with a purple colour. FIG. 5 shows the gradient in the staining of beads composed of a base polymer containing between 10 and 80% MAA that has been derivatized with hydroxamate groups, as well as the lack of staining for the underivatized 80% MAA beads (extreme right sample of beads in FIG. 3). The capacity of the hydroxamate-derivatized beads (from a 63% MAA base polymer) to inhibit the activity MMP-2 compared to underivatized beads is shown in FIG. 6. Before incubation with MMP-2 for 90 minutes at room temperature, HX and control beads were swollen in Tris-HCl/Ca buffer for 2 hours to eliminate any effects due to absorption. After pH adjustment with NaOH (to 7.6), the supernatant was incubated with FITC-gelatin for 60 minutes in the dark. MMP-2 activity was proportional to the intensity of solution fluorescence produced by the by-products of FITC-gelatin degradation.

### Example 2

### Bulk Modification

Polyacrylates may be derivatized via a nucleophilic displacement reaction by hydroxylamine in solution, yielding bulk modified, hydroxamate-containing copolymers. Poly(methylacrylate) was dissolved in DMF at approximately 10% wt/vol and the solution was placed in a sealed reactor and purged with dry, N₂ gas. The solution was heated to 45° C. and a 100% molar excess (relative to polymeric ester content) of hydroxylamine and 300% molar excess of N-methyl morpholine were added. The solution was stirred and the reaction was continued for 24 hr. The solution was cooled and the polymer was precipitated in a CaCl₂ solution. The polymer precipitate was then washed with 1 N HCl and deionized water before drying at 55° C.

### Example 3

### Hydroxamate Monomer Synthesis

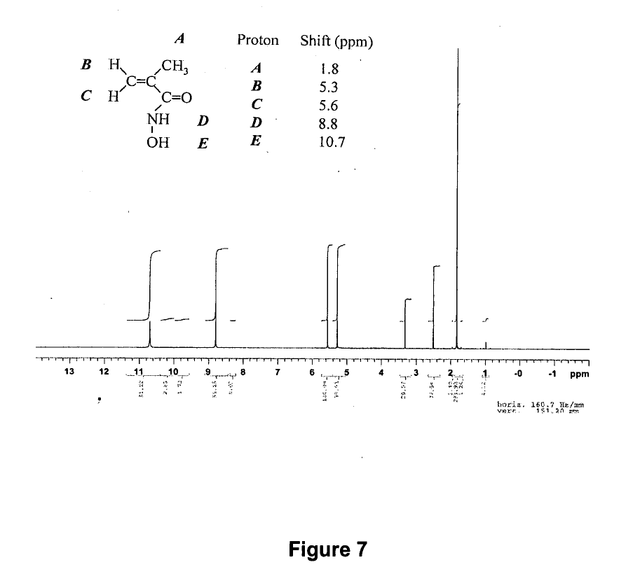

Methacrylic acid monomer was dissolved in a suitable solvent (e.g. chloroform, diethyl ether) at 7% wt/vol and 0° C. A 25% molar excess of 4-methyl morpholine and 25% molar excess of chloroformate (relative to monomer carboxylic acid content) were added to the monomer solution with stirring. The reaction proceeded for 15 min. at 0° C., then the solution was filtered. The filtrate was added to a 25% molar excess of hydroxylamine in water solution and the combined solution was stirred at room temperature for 1 hr. After completion of the reaction, a solution of 0.05M NaOH was added to the reaction mixture. The aqueous layer was then separated from the organic phase and extracted three times with fresh organic solvent. The organic layer was extracted twice with 0.05 M NaOH and all of the aqueous volumes were combined. The aqueous raw monomer solution was dried in a freeze-dryer, leaving a white tacky solid. The raw product was then purified using silica gel chromatography (thin layer or column) with ethyl acetate/methanol or diethyl ether/methanol as the eluting solvent system. The column-purified monomer was then further purified by recrystallization from a 75/25 (vol/vol) toluene/chloroform solution to yield a colourless crystalline solid. Monomer purity was evaluated by NMR spectroscopy in d₆-DMSO (FIG. 7) and found to be approximately 95 mol %.

The ferric hydroxamate test was performed on the raw, derivatized monomer. The monomer was dissolved in 0.1 M HCl, several drops of 5 wt % FeCl₃ were added and the solution immediately turned dark burgundy confirming the presence of hydroxamate functionality. Performing the test on underivatized MAA resulted in no detectable colour change. In addition, the MMP inhibiting capacity of the purified monomer was demonstrated.

### Example 4

### Modulation of MMP-8 Concentration and Activity in Buffer Solution by HX Beads

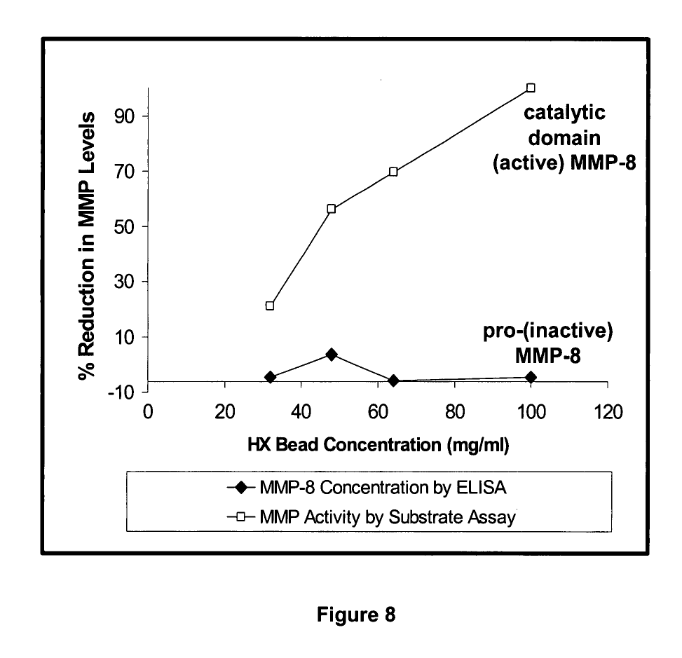

HX bead dose response curves were generated with both pro-(inactive) MMP-8 and catalytic domain (active) MMP-8 enzyme solutions. Pro-MMP-8 (15 ng/mL, R&D Systems) and catalytic domain MMP-8 (100 U/mL, Biomol International) solutions were incubated with four different doses of HX beads (32, 48, 64 and 100 mg/mL) for 1.5 h at room temperature. Following the bead incubations, pro-MMP-8 supernatants were assayed for total MMP-8 concentration using an enzyme linked immunosorbent assay (ELISA; R&D Systems). To measure MMP activity, catalytic domain MMP-8 supernatants were added to a chromogenic, broad-spectrum MMP substrate (1 mM, Biomol International). Substrate digestion was monitored by measuring optical density (absorbance) over time for 30 min and MMP activity estimated from the slope of the absorbance vs. time curve. Reductions in MMP levels for the two enzyme solutions as a function of HX bead concentration are shown in FIG. 8. MMP-8 catalytic domain demonstrated a bead-dose dependent reduction in MMP activity ranging from 24-100%. In contrast, pro-MMP-8 concentrations were not reduced. These differences in MMP reductions for the two MMP-8 forms demonstrate that HX beads have an affinity for the active form of MMP-8 compared to the inactive form.

### Example 5

### Modulation of MMP-8 Concentration and Activity in Human Chronic Wound Fluid by HXBeads

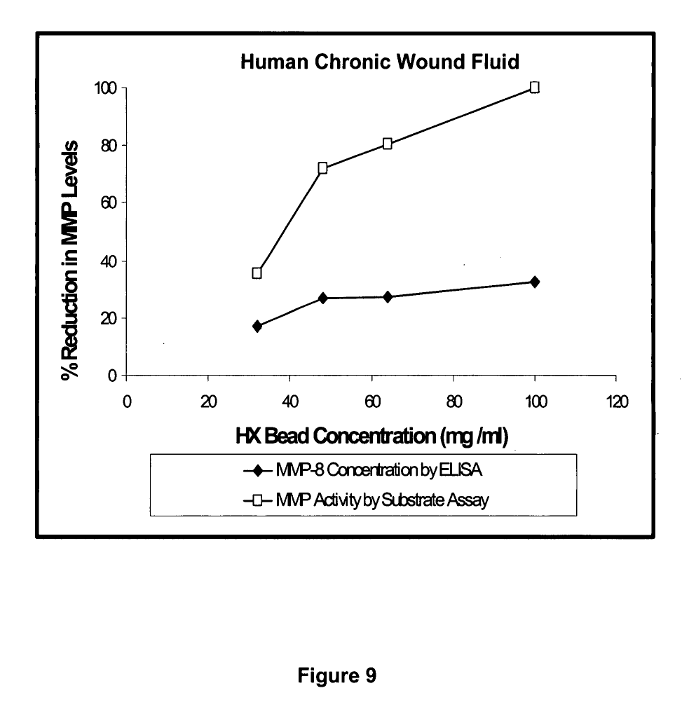

A subsequent bead-dose experiment was performed with human chronic wound fluid exudate, which contains a mixture of pro and active forms of MMP-8. Following bead incubations, wound fluid supernatants were analyzed for both MMP-8 concentration and MMP activity as described above. The HX bead dose response curves for this experiment are shown in FIG. 9. Human wound fluid exhibited a dose dependent reduction in MMP activity ranging from 36-100%, while MMP-8 concentration changed by only 17-32%. These results are consistent with those of Example 1. High reductions in MMP activity on the substrate assay are observed, reflective of the active, bead-bound component of MMP-8 in the wound fluid sample, while the inactive constituent of MMP-8 remains unaffected as seen by the modest reduction in MMP-8 concentration by ELISA.

### Example 6

### Inhibitory Activity of HXBeads Towards Several Subclasses of MMPs

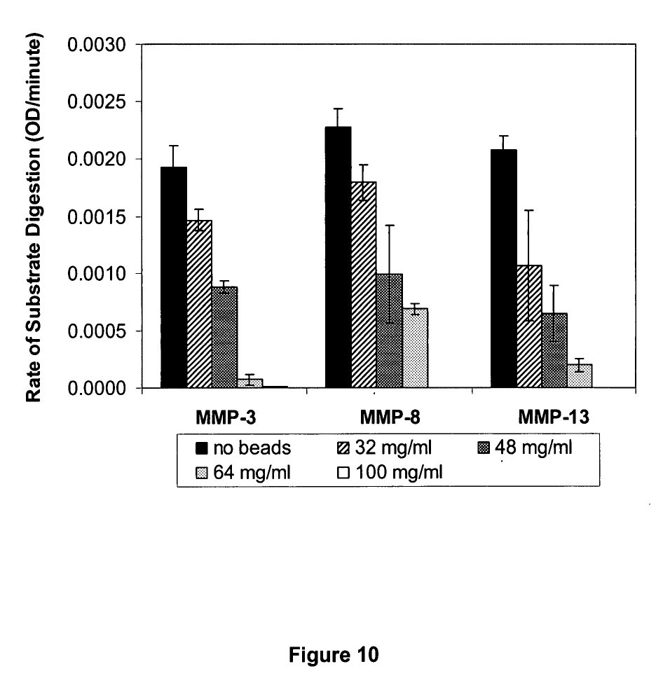

Solutions of MMP-3, -8 and -13 catalytic domains (Biomol International) were prepared at 100 U/mL. Separate aliquots were incubated with four different doses of HX beads (32, 48, 64 and 100 mg/mL) for 1.5 h at room temperature. Following the bead incubations, solution MMP activities were determined using the chromogenic substrate assay. The relative rates of substrate digestion as a function of HX bead concentration are illustrated in FIG. 10 and demonstrate a similar range of MMP inhibition (from 21-100%) for the different MMP subclasses.

### Example 7

### Modulation of MMP Activity in Human Arthritic Synovial Fluid by HXBeads

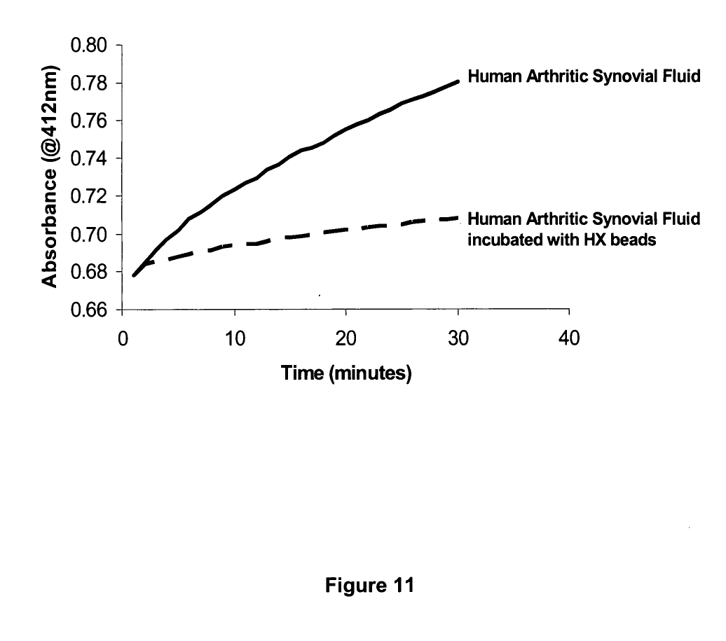

Samples of human arthritic synovial fluid, which contain a mixture of active MMPs, were incubated with 100 mg/mL of HX beads and the inhibition of MMP activity subsequently measured with the chromogenic substrate assay. As illustrated in FIG. 11, synovial fluid MMP activity is reduced by 74% following HX bead incubation.

### Example 8

### Effect of MMP-3 Activation on HXBead Efficacy

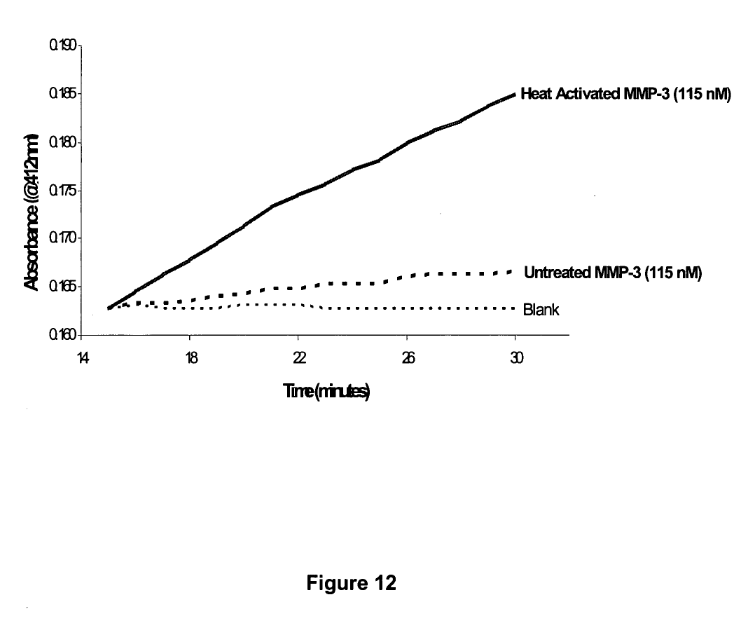

Full length human MMP-3 (115 nM, Biomol International) was activated by incubating the enzyme solution at 55° C. for 1 h [Koklitis et al., 1991]. MMP solution activity was determined prior to and following the activation procedure using the chromogenic substrate assay. As illustrated in FIG. 12, untreated MMP-3 has minimal activity towards the substrate. Following treatment there is a significant increase in the slope of the absorbance vs. time curve (>400%) indicative of successful activation.

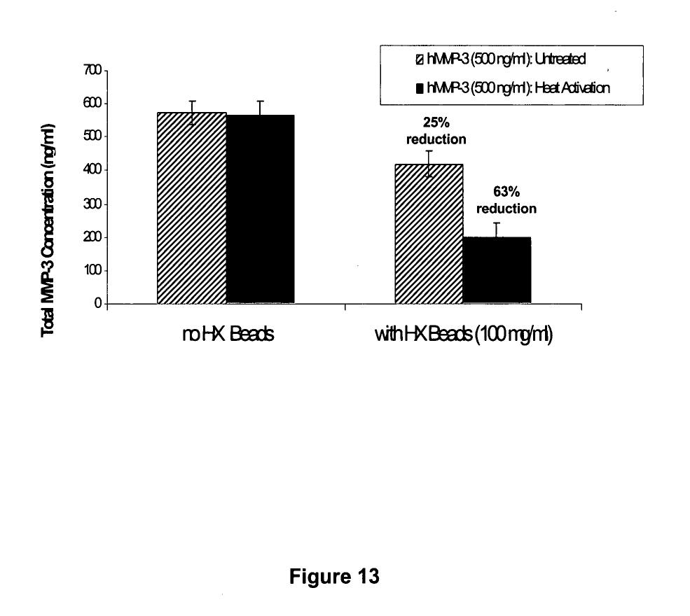

In order to determine the effect of MMP-3 activation on HX bead efficacy, samples of heat activated and untreated MMP-3 (500 ng/mL) were incubated with 100 mg/mL of HX beads for 1.5 h at room temperature. Following the incubations, supernatants were diluted 1:50 in assay diluent and analyzed by ELISA for total MMP-3 concentration. FIG. 13 shows the resulting reductions in MMP-3 concentration for the two treatment groups. A 63% reduction in concentration was achieved for the activated enzyme compared to 25% for the untreated group. This difference in MMP inhibition illustrates preferential HX bead binding towards the active form of MMP-3.

### Example 9

### Effect of MMP-8 Activation on HX Bead Efficacy

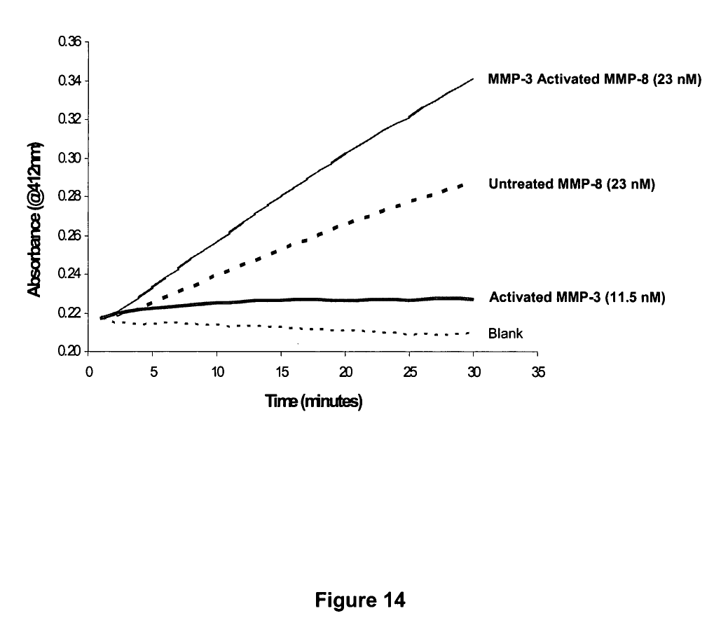

Full-length, recombinant human MMP-8 (23 nM) was activated by incubating the enzyme solution with a 2:1 molar ratio of heat activated MMP-3 for 12 h at 37° C. [Knauper et al., 1993]. MMP solution activity was determined prior to and following the activation procedure using the chromogenic substrate assay. As illustrated in FIG. 14, untreated MMP-8 is partially active. Treatment with MMP-3 increases the slope of the absorbance vs. time curve by 72%, indicating a significant increase in the proportion of active form MMP-8 with MMP-3 treatment. Notably, this increase in solution MMP activity exceeds the contribution from the added MMP-3 as illustrated by the relatively low slope of MMP-3 absorbance vs. time curve.

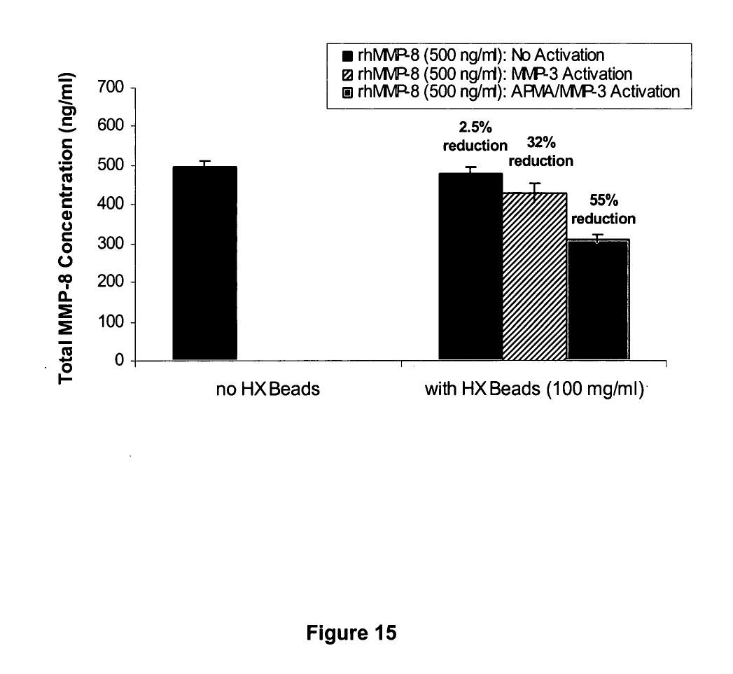

In order to determine the effect of MMP-8 activation on HX bead efficacy, samples of MMP-3 activated and untreated MMP-8 (500 ng/mL) were incubated with 100 mg/mL of beads for 1.5 h at room temperature. Following the incubations, supernatants were diluted 1:50 in assay diluent and analyzed by ELISA for total MMP-8 concentration. FIG. 15 shows the resulting reduction in MMP-8 concentration for the two treatment groups. A 32% reduction in concentration was achieved for the activated enzyme compared to 2.5% for the untreated group reflecting a preferential HX bead binding towards the activated MMP-8.

A second MMP-8 activation protocol was devised which combined the MMP-3 activation procedure described above with the addition of 1 nM APMA, a well established MMP activating reagent, for 12 h, 37° C. [Suzuki et al., 1990]. The extent of MMP-8 activation via this protocol was not assessed on the chromogenic substrate assay due to interference of the APMA with substrate digestion.

The effect of combined MMP-3/APMA activation of MMP-8 on HX bead efficacy was again determined using ELISA. As shown in FIG. 15, a 55% reduction in MMP-8 concentration was achieved following bead incubation with this activation scheme.

Although the invention describes and illustrates a preferred embodiment of the invention, it is to be understood that the invention is not so restricted and includes all alternative embodiments thereof.

### REFERENCES

Arner E C. (2002) Aggrecanase-mediated cartilage degradation. Curr. Opin. Pharmacol., 2(3), 322-9.
Ashley R. and the SDD Clinical Research Team. (1999) Clinical trials of a matrix metalloproteinase inhibitor in human periodontal disease. Ann. New York Acad. Sci., 878, 159-79.
Balbin M, Fueyo A, Knauper V, Penda A M, Lopez J M, Jimenez M G, Murphy G, Lopez-Otin C. (1998) Collagenase 2 (MMP-8) expression in murine tissue-remodeling processes. Analysis of its potential role in postpartum involution of the uterus. J. Biol. Chem., 273, 23959-6.
Chakraborti S, Mandal M, Das S, Mandal A, Chakraborti T. (2003) Regulation of matrix metalloproteinases: An overview. Mol. Cell. Biochem., 253(1-2), 269-85.
Chen, J. J., Y. P. Zhang, S. Hammond, N. Dewdney, T. Ho et al. (1996) Design, synthesis, activity & structure of a novel class of matrix metalloproteinase inhibitors containing a heterocyclic P-2′-P-3′ amide bond isostere. Biorg. Med. Chem. Lett., 123(6), 1601-1606.
Domb et al. (1992) Method of making hydroxamic acid polymers from primary amide polymers, U.S. Pat. No. 5,128,420.
Drummond A. et al. (1999) Preclinical and clinical studies of MMP inhibitors in cancer. Ann. New York Acad. Sci., 878, 159-79.
Duewell St. et al. (1991) Nonionic polyethylene glycol-ferrioxamine as a renal magnetic contrast agent. Investigative Radiology, 26(1), 50-57.
Feldhoff P. (1992) Comparison of coupling procedures for development of affinity membranes:optimization of the CDI method in Techniques in Protein Chemistry III. Academic Press, London, 151-160.
Galis Z S, Khatri J J. (2002) Matrix metalloproteinases in vascular remodeling and atherogenesis: the good, the bad, and the ugly. Circ. Res., 90(3), 251-62
Gehlbach P L, Purple R L, Hallaway P E, Hedlund B E (1993) Polymer conjugation reduces deferoxamine induced retinopathy in an albino rat model. Invest. Ophthalmol. Vis. Sci., 34(10), 2871.
George S. J. (2000) Therapeutic potential of matrix metalloproteinase inhibitors in atherosclerosis. Expert Opin Investing Drugs, 9, 993-1007.
Hallaway P., Eaton J., Panter S., Hedlund B. (1989) Modulation of deferoxamine toxicity and clearance by covalent attachment to biocompatible polymers. Proc. Nat.'l Acad. Sci., 86 (24), 10108-12.
Holopainen J M, Moilanen J A, Sorsa T, Kivela-Rajamaki M, Tervahartiala T, Vesaluoma M H, Tervo T M. (2003) Activation of matrix metalloproteinase-8 by membrane type 1-MMP and their expression in human tears after photorefractive keratectomy. Invest Ophthalmol. Vis. Sci., 44(6), 2550-6.
Horowitz, D., Margel S., Shimoni T., (1985) Iron detoxification by haemoperfusion through deferoxamine-conjugated agarose-polyacrolein microsphere beads. Biomaterials, 6, 9-16.
Huffman K. (1989) Carboxy hydroxamic acid polymers and their use as detergent additives, U.S. Pat. No. 4,874,539.
Iskander et al. (2000) Synthesis and Copolymerization of methacryloyl hydroxamic acids. Journal of Applied Polymer Sciences, 78, 751-758.
Kamble K. and Patkar D. (1994) Synthesis and characterization of hydroxamic acid resin of poly(4-vinyl-2-carboxybenzophenone). Journal of Applied Polymer Science, 52, 1361-1364.
Kern W. and Schulz R. (1957) Synthetische makromolekulare stoffe mit reactiven gruppen, Angewandte chemie, 69 (5), 153-188.
Knauper V, Murphy G, Tschesche H. (1996) Activation of human neutrophil procollagenase by stromelysin 2. Eur. J. Biochem., 235(1-2), 187-91.
Knauper V, Wilhelm S M, Seperack P K, DeClerck Y A, Langley K E, Osthues A, Tschesche H. (1993) Direct activation of human neutrophil procollagenase by recombinant stromelysin. Biochem. J., 295 (Pt 2), 581-6.
Knauper V, Kramer S, Reinke H, Tschesche H. (1990) Characterization and activation of procollagenase from human polymorphonuclear leucocytes. N-terminal sequence determination of the proenzyme and various proteolytically activated forms. Eur. J. Biochem., 189(2), 295-300.
Koklitis P A, Murphy G, Sutton C, Angal S. (1991) Purification of recombinant human prostromelysin. Studies on heat activation to give high-Mr and low-Mr active forms, and a comparison of recombinant with natural stromelysin activities. Biochem. J., 276(Pt 1), 217-21.
Lewis, E. J., J. Bishop, K. M. K. Bottomley, D. Bradshaw, M. Brewster et al. (1997) Ro 32-3555, an orally active collagenase inhibitor, prevents cartilage breakdown in vitro and in vivo. Br. J. Pharmacol., 121, 540-6.
Lijnen H R. (2004) Metalloproteinase in development and progression of vascular disease. Pathophysiol. Haemst. Thromb., 33, 275-81.
Lobmann R, Schultz G, Lehnert H. (2005) Proteases and the diabetic foot syndrome: mechanisms and therapeutic implications. Diabetes Care, 28(2), 461-71.
Lobmann R, Ambrosch A, Schultz G, Waldmann K, Schiweck S, Lehnert H. (2002) Expression of matrix-metalloproteinases and their inhibitors in the wounds of diabetic and non-diabetic patients. Diabetologia, 45(7), 1011-6.
Panter S., Braughler J., Hall E. (1992) Dextran-coupled deferoxamine improves outcome in a murine model of head injury. Journal of Neurotrauma, 9(1), 47-53.
Parks W., Sudbeck B., Doyle G. and Saariahlo-Kere U. (1998) Matrix metalloproteinases in tissue repair in Matrix Metalloproteinases. Editors:William C. parks and Robert P. Mecham, Academic Press, London, 263-97.
Prescott M. et al. (1999) Effect of matrix metalloproteinase inhibition on progression of atherosclerosis and aneurysm in LDL receptor-deficient mice over-expressing MMP-3, MMP-12, and MMP-13 and on restenosis in rats after balloon injury. Ann. New York Acad. Sci., 878, 159-79.
Marini S, Fasciglione G F, Monteleone G, Maiotti M, Tarantino U, Coletta M. (2003) A correlation between knee cartilage degradation observed by arthroscopy and synovial proteinases activities. Clin. Biochem., 36(4), 295-304.
Nwomeh B C, Liang H X, Cohen I K, Yager D R. (1999) MMP-8 is the predominant collagenase in healing wounds and nonhealing ulcers. J. Surg. Res.; 81(2), 189-95.
Sandy J D, Verscharen C. (2001) Analysis of aggrecan in human knee cartilage and synovial fluid indicates that aggrecanase (ADAMTS) activity is responsible for the catabolic turnover and loss of whole aggrecan whereas other protease activity is required for C-terminal processing in vivo. Biochem. J., 358(Pt 3):615-26.
Schouteden F. (1958) On the conversion of amidoxime groups into hydroxamic acid groups in polyacrylamidoximes. Makromolekulare Chemie, 27(3), p. 246-255.
Saarialho-Kere U. (1998) Patterns of matrix metalloproteinase and TIMP expression in chronic ulcers. Arch. Dermatol. Res., 290, S47-S54.
Shalinsky D. et al. (1999) Broad antitumour and antiangiogenic activities of AG3340, a potent and selective MMP inhibitor undergoing advanced oncology clinical trials. Ann. New York Acad. Sci., 878, 159-79.
Sparapany J., Fong D., Fu J. (1989) Polyhydroxamic acid polymers from polyacrylic acids, U.S. Pat. No. 4,868,248.
Stricklin G P, Jeffrey J J, Roswit W T, Eisen A Z. (1983) Human skin fibroblast procollagenase: mechanisms of activation by organomercurials and trypsin. Biochemistry, 22(1), 61-8.
Suzuki K, Enghild J J, Morodomi T, Salvesen G, Nagase H. (1990) Mechanisms of activation of tissue procollagenase by matrix metalloproteinase 3 (stromelysin). Biochemistry, 29(44), 10261-70.
Tchetverikov I, Lohmander L S, Verzijl N, Huizinga T W, TeKoppele J M, Hanemaaijer R, DeGroot J. (2005) MMP protein and activity levels in synovial fluid from patients with joint injury, inflammatory arthritis, and osteoarthritis. Ann. Rheum. Dis., 64(5):694-8.
Tchetverikov I, Ronday H K, Van El B, Kiers G H, Verzijl N, TeKoppele J M, Huizinga T W, DeGroot J, Hanemaaijer R. (2004) MMP profile in paired serum and synovial fluid samples of patients with rheumatoid arthritis. Ann. Rheum. Dis., 63(7), 881-3.
Thompson R. and Baxter B. (1999) MMP inhibition in abdominal aortic aneurysms: rationale for a prospective randomized clinical trial, Ann. New York Acad. Sci., 878, 159-79.
Trengove N J, Stacey M C, MacAuley S, Bennett N, Gibson J, Burslem F, Murphy G, Schultz G. (1999) Analysis of the acute and chronic wound environments: the role of proteases and their inhibitors. Wound Repair Regen., 7(6), 442-52.
Van Wart H E, Birkedal-Hansen H. (1990) The cysteine switch: a principle of regulation of metalloproteinase activity with potential applicability to the entire matrix metalloproteinase gene family. Proc. Natl. Acad. Sci. USA., 87(14), 5578-82.
Vernon F. and Zin W. (1981) Chelating ion exchangers containing N-substituted hydroxylamine functional groups Part 6. Sorption and separation of gold and silver by a polyhydroxamic acid. Analytica Chimica Acta., 123, 309-313.
Vernon F. and Eccles H. (1976) Chelating ion-exchangers containing N-substituted hydroxylamine functional groups Part III. Hydroxamic acids. Anal. Chim. Acta, 82.
Vernon F. and Eccles H. (1976) Chelating ion-exchangers containing N-substituted hydroxylamine functional groups Part IV. Column separations on a hydroxamic acid resin. Anal. Chim. Acta, 83.
Wenk J. et al. (2001) Selective pick-up of increased iron by deferoxamine-coupled cellulose abrogates the iron-driven induction of matrix-degrading metalloprteinasel and lipid peroxidation in human dermal fibroblasts in vitro: a new dressing concept. The Journal of Investigative Dermatology, 116 (6), 833-839.
Witte M. et al. (1998) Metalloproteinase inhibitors and wound healing: a novel enhancer of wound strength. Surgery, 124 (2), 464-470.
Winston A., Varaprasad P., Metterville J., Rosenkrantz H. (1985) Evaluation of polymeric hydroxamic acid iron chelators for treatment of iron overload. Journal of Pharmacology and Experimental Therapeutics, 232 (3), 644-649.
Winston A., Varaprasad D. (1986) Polymeric iron chelators, U.S. Pat. No. 4,613,616.
Woessner Jr. J. (1998) The matrix metalloproteinase family in Matrix Metalloproteinases, Editors:William C. parks and Robert P. Mecham, Academic Press, London, 1-13.
Wojtowicz-Praga, S., J. Low, J. Marshall, E. Ness, R. Dickson et al. (1996) Phase 1 trial of a novel matrix metalloproteinase inhibitor batimastat (BB-94) in patients with advanced cancer. Invest. New Drugs, 14, 193-202.
Wysocki A B, Staiano-Coico L, Grinnell F. (1993) Wound fluid from chronic leg ulcers contains elevated levels of metalloproteinases MMP-2 and MMP-9. J. Invest. Dermatol., 101(1), 64-8.
Xu Y., Ogawa H., Kanoh S., Motoi M. (1999) Preparation and characterization of polyoxetane resins anchoring the pendant space-separated hydroxamic acid residue. Polymer Journal, 31, 143-149.
Yager D R, Zhang L Y, Liang H X, Diegelmann R F, Cohen I K. (1996) Wound fluids from human pressure ulcers contain elevated matrix metalloproteinase levels and activity compared to surgical wound fluids. J. Invest. Dermatol., 107(5), 743-8.
Yoshihara Y, Nakamura H, Obata K, Yamada H, Hayakawa T, Fujikawa K, Okada Y. (2000) Matrix metalloproteinases and tissue inhibitors of metalloproteinases in synovial fluids from patients with rheumatoid arthritis or osteoarthritis. Ann. Rheum. Dis., 59(6), 455-61.

1. A therapeutic polymer containing a hydroxamate group containing a hydroxamate group 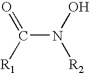
wherein R₁ is a linear, branched or crosslinked polymer or a linker group connecting the hydroxamate group to a polymer; and R₂ is selected from the group consisting of hydrogen, alkyl group, alkyl halide, alkene group, aryl group, heteroaryl, amino acid, peptide, (oligo)ether, heterocyclic group, polymer, polymerizable group, carboxylic acid, ester, amide, epoxide, ketone, aldehyde, alcohol for binding zinc-containing enzymes.

2. A therapeutic polymer as claimed in claim 1 for binding biological species containing divalent metal ions.

3. A therapeutic polymer as claimed in claim 2 for binding zinc-containing proteases.

4. A therapeutic polymer as claimed in claim 2 for binding matrix metalloproteinase.

5. A therapeutic polymer as claimed in claim 4 for binding the active forms of matrix metalloproteinases.

6. A therapeutic polymer as claimed in claim 4 for binding the inactive form of matrix metalloproteinases.

7. A therapeutic polymer as claimed in claim 4 that has a higher affinity for binding the active forms of matrix metalloproteinases in comparison to the inactive form of matrix metalloproteinases.

8. A therapeutic polymer containing a hydroxamate group as claimed in claim 1 for binding active forms of a matrix metalloproteinase in multi-protein physiologic solutions.

9. A therapeutic polymer as claimed in claim 8 wherein said matrix metalloproteinase has been activated by a physiologic activator.

10. A therapeutic polymer as claimed in claim 9 wherein said physiologic activator is a reactive oxygen species released from activated inflammatory cells.

11. A therapeutic polymer as claimed in claim 9 wherein said physiologic activator is a proteolytic agent.

12. A therapeutic polymer as claimed in claim 11 wherein said proteolytic agent is selected from one of the following groups of proteolytic enzymes: plasminogen activators, matrix metalloproteinases, serine proteinases and bacterial proteinases.

13. A therapeutic polymer as claimed in claim 12 wherein said proteolytic enzyme is selected from the group consisting of: tissue-type plasminogen activator, urokinase type plasminogen activator, MMP-3, MMP-7, MMP-10, MMP-14, plasmin, trypsin, chymotrypsin, cathepsin G.

14. A therapeutic polymer as claimed in claim 8 wherein said matrix metalloproteinase has been activated by a non-physiologic activator.

15. A therapeutic polymer as claimed in claim 14 wherein said non-physiologic activator has been selected from one of the following groups of chemical reagents: organomercurials, sulfhydryl alkylating agents, disulfide compounds, conformational pertubants, heavy metals ions.

16. A therapeutic polymer as claimed in claim 15 wherein said chemical reagent has been selected from the group consisting of: aminophenyl mercuric acetate, N-ethylmaleimide, oxidized glutathione, sodium docecyl sulfate, sodium thiocyanate, Au(I) compounds, Hg(II) compounds.

17. A medical device for the inhibition of matrix metalloproteinases which comprise a therapeutic polymer containing a hydroxamate group as claimed in claim 1.

18. A medical device as claimed in claim 17 wherein said polymer was synthesized by surface modification of cross-linked polymethacrylic acid-co-methyl methacrylate beads.

19. A surface modified derivatizable polymer containing a hydroxamate group as claimed in claim 1.

20. The polymer of claim 19, wherein the derivatizable polymer is polymethacrylic acid-co-methyl methacrylate.

21. A hydroxamate group containing polymer as claimed in claim 1 synthesized by copolymerizing a polymerizable monomer containing a hydroxamate group with a comonomer.

22. A therapeutic polymer as claimed in claim 1 containing a derivatizable polymer with a hydroxamate containing group grafted thereon.

23. The polymer of claim 22 wherein the graft consists of hydroxamate containing monomer units ranging 1 to 1,000,000 in number.

24. A therapeutic polymer for slowing, preventing or reversing tissue remodeling and destruction comprising a therapeutic polymer containing a hydroxamate group as claimed in claim 1.

25. A therapeutic polymer for controlling inflammation comprising a therapeutic polymer containing a hydroxamate group as claimed in claim 1.

26. A therapeutic polymer for restricting cell migration comprising a therapeutic polymer containing a hydroxamate group as claimed in claim 1.

27. Beads for slowing, preventing or reversing tissue remodeling and destruction comprising a therapeutic polymer containing a hydroxamate group as claimed in claim 1.

28. Beads for controlling inflammation comprising a therapeutic polymer containing a hydroxamate group as claimed in claim 1.

29. Beads for restricting cell migration comprising a therapeutic polymer containing a hydroxamate group as claimed in claim 1.

30. A wound care product which comprises a therapeutic polymer as claimed in claim 1 incorporated into a substrate.

31. A wound care product as claimed in claim 30 wherein said substrate is a dressing, a cream or an ointment.

32. A wound care product comprising a thermoreversible gel in which hydroxamate beads as claimed in claim 27 have been incorporated.

33. A wound care product as claim in claim 31 wherein said gelable composition comprises a copolymer and a solvent, the copolymer having the structure A(B)n, wherein A is soluble in the solvent, B is convertible between soluble and insoluble in the solvent depending on an environmental condition, and n is greater than 1, the composition being convertible from liquid to gel under an environmental condition where B is insoluble.

34. A wound care product as claimed in claim 33 wherein said environmental condition is selected from the group consisting of temperature, pH, ionic strength, and a combination thereof.

35. A wound care product as claimed in claim 33 wherein said environmental condition is temperature.

36. A wound care product as claimed in claim 33 wherein A is selected from the group consisting of polyethylene glycol (PEG), polyvinyl pyrrolidone, polyvinyl alcohol, polyhydroxyethylmethacrylate, and hyaluronic acid.

37. A wound care product as claimed in claim 31 wherein B is selected from the group consisting of poly-N-isopropyl acrylamide (PNIPAAm), methyl celluloses, poly(ethylene glycol vinyl ether-co-butyl vinyl ether), polymers of N-alkyl acrylamide derivatives, poly(amino acids)s, poly(methacryloy L-alanine methyl ester), poly(methacyloy L-alanine ethyl ester) and nitrocellulose.

38. A wound care product as claimed in claim 31 wherein the copolymer is present in the solvent at a level of from 5% to 50% by weight.

39. A wound care product as claimed in claim 31 wherein the copolymer is present in the solvent at a level of from 10% to 25% by weight.

40. A wound care product as claimed in claim 31 wherein n is 2, 4 or 8.

41. A wound care product as claimed in claim 31 wherein n is greater than or equal to 4.

42. A wound care product as claimed in claim 31 wherein A is polyethyleneglycol (PEG).

43. A wound care product as claimed in claim 31 wherein B is poly-N-isopropyl acrylamide (PNIPAAm).
